(19) United States
(12) Patent Application Publication
(10) Pub. No.: US 2004/0086581 A1
(43) Pub. Date: May 6, 2004

(54) Bio-energetic joint and arthritis pain formula

(76) Inventor: Edwin B. Jones, Redding, CA (US)

Correspondence Address: LANNY M. ENGLUND, 1472 Ashover Drive, Bloomfield Hills, MI, 48304, US

(21) Appl. No.: 10/288,969

(22) Filed: Nov. 6, 2002

(51) Int. Cl. A61K 38/48; A61K 35/78; A61K 38/39; A61K 31/375

(52) U.S. Cl. 424/756; 424/764; 424/769; 424/770; 424/766; 514/2; 514/62; 514/474; 424/94.64

(57) ABSTRACT

A composition of matter generally relating to a joint and arthritis pain formula with a unique and optimized blending of ingredients to promote the healing and relief of arthritis symptoms, with an added chelated and bio-energized delivery system to insure proper absorption, assimilation, and digestion.

### BACKGROUND OF THE INVENTION

1. Field of the Invention

The present invention relates generally to a joint and arthritis pain formula and more particularly to a unique and optimized blending with certain ingredients that have never before been combined into one single formula to promote healing and relieve arthritis symptoms. An advanced chelated and bio-energized delivery system has been added to insure proper absorption, assimilation, and digestion.

Arthritis is a disease of epidemic proportions and is thought to affect more than 50 million Americans. It is generally accepted to be the leading cause of joint movement limitations and disability while receiving a great deal of research and medical attention.

Osteoarthritis is the most common form of joint disease. It is generally considered to be due to “wear and tear” of the joints leading to damage of the joint surfaces which gives pain on movement. The condition is very common in older groups, but can affect younger people as well. There are many factors that influence its development, including a family history and previous damage to the joint through injury or surgery.

2. Description of the Related Art

There are hundreds of drugs, procedures, medical aids, and devices directed at coping with the manifestations of arthritis as a disease. Given this degree of complexity, certainly no one agent alone could ever be expected to manage or cure arthritis in its entirety. New agents continually take their place in the spectrum for treating arthritis. However, there has been a long felt need for the optimum combination of ingredients with these agents to effectively and efficiently treat arthritis all in one optimized product formula. The subject patent provides a unique optimized blended formula to satisfy that need.

Relevant prior art treatments for rheumatoid arthritis have included the synthesis of cortisone from soy beans, synthesized physostigmine, 2-chloro-deoxyadenosine, aspirin, acetaminophen, ibuprofen, peptides, compositions comprising methotrexate and pentostatin, antiangiogenic drug, alkyl hydrogen fumerates, triazolopyridine anti-inflammatory agents, benzimidazole anti-inflammatory compounds, heterocyclic hydroxamic acid, and many others. Remedies include candles, bee lip balm, and bee stings, as well as extreme cold combined with exercise for treating arthritis and osteoarthritis.

U.S. Pat. No. 6,465,473 to Bonner, Jr., et al describes a method for treatment of reactive arthritis. Patent No. GB 2,374,533 teaches an electro-therapy device. U.S. Pat. No. 2,143,143 discloses peptides designed for the diagnosis and treatment of rheumatoid arthritis. Correspondingly, the U.S. Pat. No. 2,137,092 also discloses peptide derived from an antigen recognized by autoantibodies from patients with rheumatoid arthritis. U.S. Pat. No. 2,136,724 to Mohler describes methods for treating rheumatoid arthritis using IL-17 antagonists.

For the most part, the relevant prior art for the treatment of arthritis lies in pharmacological treatments with pharmaceutical patent medicines and other remedies. The inherent disadvantages and shortcomings of these approaches lie in the multiple choice variability of options and the trial and error selection criteria for targeted results with specific arthritis afflictions.

The typical arthritis patient may be totally confused with too many product claims from the pharmaceutical industry from too many product options and may not select the right product for the patient's specific needs. The quest for a common treatment and potential cure for arthritis in one formulation has always been frustrated with the prior art. Many of the shortcomings and disadvantages of the prior art formulations have now been overcome with the joint and arthritis pain formula in the present invention.

### SUMMARY OF THE INVENTION

The Bio-Energetic joint and and arthritis pain formula as described in the subject invention represents a unique formula blending with certain ingredients that have never before been combined in one formula. An advanced chelated delivery system is also combined with a “bio-energized” delivery system that actually energizes the formula making it stronger and more active.

The bio-energetic joint and arthritis pain formula of the subject invention contains, but is not limited to, at least the following ingredients including CMO-Cetyl Myristoleate, a unique natural compound valuable in arthritis conditions. Cetyl myristoleate shows promise of making an great contribution in non-infective types of arthritis. MSM, in the form of MSM ascorbate, has been added to the formulation as a detoxifier and has gained considerable recognition for its results in relieving arthritis. MSM makes cells permeable, allowing water and nutrients to flow freely into cells and allowing wastes and toxins to flow out.

Rheumatoid arthritis is an inflammatory synovial disease thought to involve T cells reacting to an antigen within the joint. Type II collagen is a major protein in articular cartilage and is a potential autoantigen in this disease. Hydrolyzed collagen, as derived from oral type IT collagen, collagen gelatin, or Knox gelatin, may be used in the subject invention formula. Collagen is a preferable treatment because it appears to improve the health of the arthritic joint as evidenced by joint functionality over time and is non-toxic.

Glucosamine is used extensively in Europe to treat osteoarthritis. Numerous published studies document glucosamine's ability to function as a chondroprotective agent. Chondroprotective agents are compounds the body manufactures naturally in order to regenerate cartilage and healthy joint function. The subject formulation may contain Glucosamaine sulfate and Glucosumine 2 KCL.

Bromelain, as contained in the subject invention formulation, is an enzyme found in pineapples, and may help decrease the inflammation associated with arthritis. It acts primarily to help maintain homeostasis in inflammatory processes. The combination of anti-inflammatory action and protein dissolving abilities enables bromelain to do its job.

Boswellia, in the form of Boswellia Serrata, as also used in the subject formulation, is an anti-arthritic and anti-inflammatory plant-derived extract. Its anti-inflammatory actions are indicated in clinical medicine for the treatment of rheumatoid arthritis, osteoarthritis, juvenile rheumatoid arthritis, soft tissue rheumatism, gout, low back pain, myositis and fibrositis, and are attributed primarily to the presences of boswellic acids. The therapeutic action of a standardized Boswellia serrata Includes: reduction of joint swelling, restoration and improvement of blood supply to the inflamed joints, pain relief, and increased mobility. Boswellia 65% is used in the subject formulation.

Turmeric, as added to the subject invention formulation possesses anti-inflammatory properties. Tumeric has been used in Indian systems of medicine for a long time. It is listed in an Assyrian herbal dating from about 600 BC and is also mentioned by Dioscorides. Both the East and West have held its medicinal properties in high regard.

Feverfew, and Feverfew extract, as used in the subject formulation may help arthritis when it is in the painfully active inflammatory stage. It also has relevant therapeutic value in treating osteo-arthritis.

Ginger, and ginger 5% formulation, also used in addition to the subject invention could be an effective treatment for arthritis, studies have shown. One study, led by Rheumatologist Professor Ray Altman, believes the highly concentrated supplement could help reduce the pain suffered by two out of three people with arthritis.

White willow extract from white willow bark, another component of the subject formulation, has been used for centuries worldwide as a pain reliever. Willow bark contains other compounds that the body metabolizes to salicylic acid. For this reason willow bark extract acts more slowly over a longer period of time than does acetylsalicylic acid, otherwise known as aspirin does. The compound salicin from willow does not cause gastric or intestinal upset or bleeding that aspirin causes. The willow bark extract reduces inflammation and relieves swollen joints due to rheumatism and arthritis.

Hyaluronic acid (HA), a key component of collagen, protects the synovial fluid in the joints and may be helpful in building joint collagen. Bathing the cartilage is the function of synovial fluid, and its chief component is hyaluronic acid (HA), which serves as an antioxidant, lubricant, and shock absorber during weight bearing. HA, also serves to promote healthy skin texture, and eye tissues, with its anti-aging properties. Laboratory studies are now in place to incorporate the useful benefits of HA in the Bio-energetic joint and and arthritis pain formula of the subject invention.

Buffered vitamin C, manganese chelate, Lo Han Ghuo, and maltodextrin have also been added to the subject formulation for improved system absorption, assimilation, sweetening, and potency. Flavorings have been added to the subject joint and arthritis pain formula for improved taste; not limited to, but including orange and vanilla flavorings in product offering and container packaging.

Grape seed extract from Italian Grapes and Pine Bark from the Maritime trees in France are called and referred to as OPC Proanthocyandians. These (2) ingredients are for inflammation and have super antioxidant qualities for the immune system down to the cellular level, and have been added to the formula.

### DETAILED DESCRIPTION OF THE PREFERRED EMBODIMENT

The bio-energetic joint and arthritis pain formula as generally represented in the summary of the invention is combined with an advanced chelated and “bio-energized” delivery system in the preferred embodiment, thus distinguishing it over the prior art.

An ACD (advanced chelated delivery system) has been added to insure proper absorption, assimilation, and digestion. The ACD delivery system is a unique delivery that has been micro meshed (meaning finer mesh size and particle size for faster and better absorption). The ACD system is also combined with a “bio-energized” delivery system called “Vital Force” energy developed by Dr. Yury Kronn, that actually energizes the formula, making it 5-10 times stronger and more active.

The bio-energetic “vital force” technology harnesses specific formulations of bio-energy and infuses them into “substance-carriers” that dramatically enhance the treated substance's bioactivity. This process is accomplished using a plasma-based low intensity alternating magnetic fields (LIAMF) for generating bio-energy. The “substance carriers” are specifically processed trace minerals, acting as catalysts and “energy capacitors” holding energy for years. This bio-energized process increases the body's ability to absorb the joint and arthritis pain formula component ingredients of the subject invention.

The bio-energetic delivery with the “Vital Force” infusion process formula has demonstrated the ability to balance and normalize the Autonomic Nervous System (ANS) within minutes. Since the ANS is responsible for 99.9% of all life functions, improving the ANS using the bio-energized joint and arthritis pain formula delivery system could improve both physiology and quality of life.

The synergistic effect of combining nutritional supplements with the bio-energitic “Vital Force” infusion process dramatically restores the ability of the body's organs to effectively perform their functions and fully utilize nutrients, as well as speed up the biological actions of all other nutritional and therapeutic modalities.

The bio-energetic joint and arthritis pain formula of the subject invention in the preferred embodiment further comprises: CMO-Cetyl Myristoleate; MSM Ascorbate; Type II collagen in the form of collagen gelatin, Knox gelatin, or hydrolyzed collagen; Glusomine sulfate, Glucosumine 2 KCl, and Chondroitin sulfate, Bromelain; Boswellia, in the form of Boswellia Serrata; Turmeric as Turmeric 95%; Feverfew; Ginger, as Ginger 5%; White willow extract; Manganese Chelate; Sucralose; Maltodextrin; Buffered Vitamin C; and Orange flavoring.

What is claimed is:

1. A joint and arthritis pain formula comprising: CMO-Cetyl Myristoleate; MSM-Organic Sulphur; Collagen; Glucosamine; OPC Proanthocyandians; Bromelain Boswellia; Turmeric; Feverview; Ginger; White willow extract; Manganese Chelate; Sweetner; Maltodextrin; Buffered Vitamin C; and flavoring.

2. A joint and arthritis pain formula as recited in claim 1, wherein the Glucosamine is Gluscosamine sulfate.

3. A joint and arthritis pain formula as recited in claim 1, wherein the Glucosamine is Glucosamine 2 KCL.

4. A joint and arthritis pain formula as recited in claim 1, wherein the MSM is MSM (Ascorbate).

5. A joint and arthritis pain formula as recited in claim 1, wherein the Collagen source may be Collagen gelatin, Knox gelatin, or Hydrolized Collagen.

6. A joint and arthritis pain formula as recited in claim 1, further comprising an ACD (advanced chelated delivery system) that has been micro-meshed for faster and better absorption.

7. A joint and arthritis pain formula as recited in any of the preceding claims, in which a bio-energetic “Vital Force” infusion process formula using a plasma-based low intensity alternating magnetic field (LIAMF) generates the bio-energy for the pain formula.

8. A joint and arthritis pain formula comprising:

| | Product | Amt/15 g-serving |
|---|---|---|
| (a) | Hydrolized Collagen; | 4000 mg |
| (b) | MSM (Ascorbate); | 4000 mg |
| (c) | CMO; | 250 mg |
| (d) | Grape Seed, Pine Bark (OPC) | 200 mg |
| (e) | Glucosamine 2KCL; | 1000 mg |
| (f) | Bromelain 10:1 Extract; | 200 mg |
| (g) | Manganese Chelate; | 3 mg |
| (h) | Buffered Vitamin C; | 500 mg |
| (i) | Boswelia 65%; | 100 mg |
| (j) | White Willow Extract; | 100 mg |
| (k) | Tumeric 95%; | 50 mg |
| (l) | Feverfew Extract; | 100 mg |
| (m) | Ginger 5%; | 100 mg |
| (n) | Lo Han Ghuo (Sweetner) | 250 mg |
| (o) | Maltodextrin; and | 2600 mg |
| (p) | Orange Flavor. | 1547 mg |

9. A joint and arthritis pain formula as recited in claim 8, further comprising an ACD (advanced chelated delivery system) that has been micro-meshed for faster and better absorption.

10. A joint and arthritis pain formula as in either claim 8 or 9, further comprising a bio-energetic “Vital Force” infusion process formula using a plasma-based Low Intensity Alternating Magnetic Field (LIAMF) for generating bio-energy.
